(19) United States
(12) Patent Application Publication
(10) Pub. No.: US 2007/0208396 A1
(43) Pub. Date: Sep. 6, 2007

(54) Systems and methods for providing a dynamic light pad

(76) Inventors: Gary Whatcott, Holladay, UT (US); Chris Spencer, Riverton, UT (US); Scott Davis, Bountiful, UT (US); Ronald Hatch, Orem, UT (US); Larry Beardall, Sandy, UT (US); Curtis Stimpson, Tremonton, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/368,021

(22) Filed: Mar. 3, 2006

(51) Int. Cl. A61N 5/06

(52) U.S. Cl. 607/88

(57) ABSTRACT

Systems and methods for providing a light pad configured for light therapy, wherein the light pad is dynamically flexible to conform to curvatures of the human body. The light pad includes a flexible, electrical circuit board configuration, a plurality of light sources coupled to the flexible, electrical circuit board configuration, wherein the plurality of light sources are configured to provide light therapy to a patient, and an electrical circuit comprising the plurality of light sources and electrical interconnections of the flexible, electrical circuit board configuration. In at least some embodiments, the flexible, electrical circuit board configuration comprises a plurality of circuit boards that are interconnected by one or more braided cords. The circuit boards provide increased flexibility of the light pad as bending is allowed between the boards, while the braided cord provides a resilient, long-lasting, electrical connection between the independent boards.

### BACKGROUND OF THE INVENTION

1. Field of the Invention

The present invention relates to a light therapy device. In particular, the present invention relates to systems and methods for providing a light pad configured for light therapy, wherein the light pad is dynamically flexible to conform to curvatures of the human body.

2. Background and Related Art

High-powered lasers generally emit collimated, coherent light that has been used to cut and/or burn away tissue, such as in surgical techniques. More recently, low-powered light sources that do not sever or destroy tissue have been used with the intention to provide light therapy to a patient.

The light therapy is applied using a hand held light therapy device. However, while such devices are currently available, the surface area for administration of the light therapy is relatively small. As such, attempts have been made to create a device that provides light therapy across an increased surface area of a patient. However, such devices have been unable to match the contour of the patient's body, which can provide inconsistent light therapy treatment.

Thus, while techniques currently exist that are used to provide light therapy to a patient, challenges still exist. Accordingly, it would be an improvement in the art to augment or even replace current techniques with other techniques.

### SUMMARY OF THE INVENTION

The present invention relates to a light therapy device. In particular, the present invention relates to systems and methods for providing a light pad configured for light therapy, wherein the light pad is dynamically flexible to conform to curvatures of the human body.

Implementation of the present invention takes place in association with a light therapy pad comprising a flexible, electrical circuit board configuration, a plurality of light sources coupled to the flexible, electrical circuit board configuration, wherein the plurality of light sources are configured to provide light therapy to a patient, and an electrical circuit comprising the plurality of light sources and electrical interconnections of the flexible, electrical circuit board configuration.

The light therapy pad includes a housing configured to receive the flexible, electrical circuit board configuration. In at least some implementations, the housing includes a polymer insulating material and a thermal conductive component or layer. The conductive component acts as a heat sink, spreading out any heat obtained from electrical components of the flexible, electrical circuit board configuration.

In some implementations, the flexible, electrical circuit board configuration includes a single flexible PC board. In other implementations, the flexible, electrical circuit board includes a plurality of boards.

In some implementations, the flexible, electrical circuit board configuration comprises a plurality of circuit boards that are interconnected by one or more stranded or braided cords. The circuit boards provide increased flexibility of the light pad as bending is allowed between the boards. The cords provide resilient, long-lasting, electrical connections between the independent boards.

Implementations of the present invention embrace utilization of a light therapy pad to administer light therapy treatment. Examples of light therapy treatment in accordance with the present invention include use of the systems and/or methods of the present invention for the treatment of an injury, tension headaches, chronic pain, migraine headaches, tension headaches, atypical facial pain, TMJ disorders, occipital neuralgia, neck-shoulder pain, fibromyalgia, medial epicondylitis, lateral epicondylitis, carpal tunnel syndrome, osteoarthritis, rheumatoid arthritis, pain and/or stiffness associated with arthritis, muscle spasm, costochondritis, spondylitis, low back strain, joint pain, sciatica, achilles tendonitis, ankle sprain, plantar fasciitis, shingles, Raynaud's Syndrome, reflex sympathetic dystrophy (also known as chronic regional pain syndrome), postherpetic neuralgia, burns, inflammation, pain, muscle spasm, wound healing, and the like. Additionally, the light therapy treatment may be administered before, during and/or after traction treatment, such as a treatment that provides lumbar and/or cervical traction to a patient.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates a dynamic light pad configured for light therapy in accordance with a representative embodiment of the present invention;

FIG. 2 illustrates a series of circuit boards manufactured for utilization in a dynamic light pad in accordance with an embodiment of the present invention;

FIG. 3 illustrates a top view of a flexible electrical circuit board configuration that comprises a plurality of circuit boards interconnected by a braided cord for utilization in a dynamic light pad in accordance with an embodiment of the present invention;

FIG. 4 illustrates a bottom view of the flexible electrical circuit board configuration of FIG. 3, which comprises a plurality of circuit boards interconnected by a braided cord for utilization in a dynamic light pad in accordance with an embodiment of the present invention;

FIG. 5 illustrates a more detailed top view of the flexible electrical circuit board configuration of FIG. 3, which comprises a plurality of circuit boards interconnected by a braided cord for utilization in a dynamic light pad in accordance with an embodiment of the present invention;

FIG. 6 illustrates a more detailed bottom view of the flexible electrical circuit board configuration of FIG. 3, which comprises a plurality of circuit boards interconnected by a braided cord for utilization in a dynamic light pad in accordance with an embodiment of the present invention;

FIG. 7 illustrates a top view of a representative top housing of a dynamic light pad in accordance with an embodiment of the present invention; and

FIG. 8 illustrates a bottom view of a representative top housing of a dynamic light pad in accordance with an embodiment of the present invention.

### DETAILED DESCRIPTION OF THE INVENTION

The present invention relates to a light therapy device. In particular, the present invention relates to systems and methods for providing a light pad configured for light therapy, wherein the light pad is dynamically flexible to conform to curvatures of the human body.

Embodiments of the present invention embrace a light therapy pad comprising a flexible, electrical circuit board configuration, a plurality of light sources coupled to the flexible, electrical circuit board configuration, wherein the plurality of light sources are configured to provide light therapy to a patient, and an electrical circuit comprising the plurality of light sources and electrical interconnections of the flexible, electrical circuit board configuration. In at least some embodiments, the light therapy pad is flexible in at least two directions.

At least some embodiments of the present invention include a light therapy pad having a housing configured to receive the flexible, electrical circuit board configuration. In some embodiments, the housing comprises a polymer insulating material. In further embodiments, the housing includes a thermal conductive component or layer that acts as a heat sink, spreading out heat obtained from electrical components of the flexible, electrical circuit board.

In some embodiments of the present invention, the flexible, electrical circuit board includes one or more flexible PC ports. In other implementations, the flexible, electrical circuit board includes a plurality of interconnected rigid boards.

In some embodiments, the flexible, electrical circuit board configuration comprises a plurality of circuit boards that are interconnected by one or more stranded and/or braided cords. The circuit boards provide increased flexibility of the light pad as bending is allowed between the boards. The braided cords provide resilient, long-lasting, electrical connections between the independent boards.

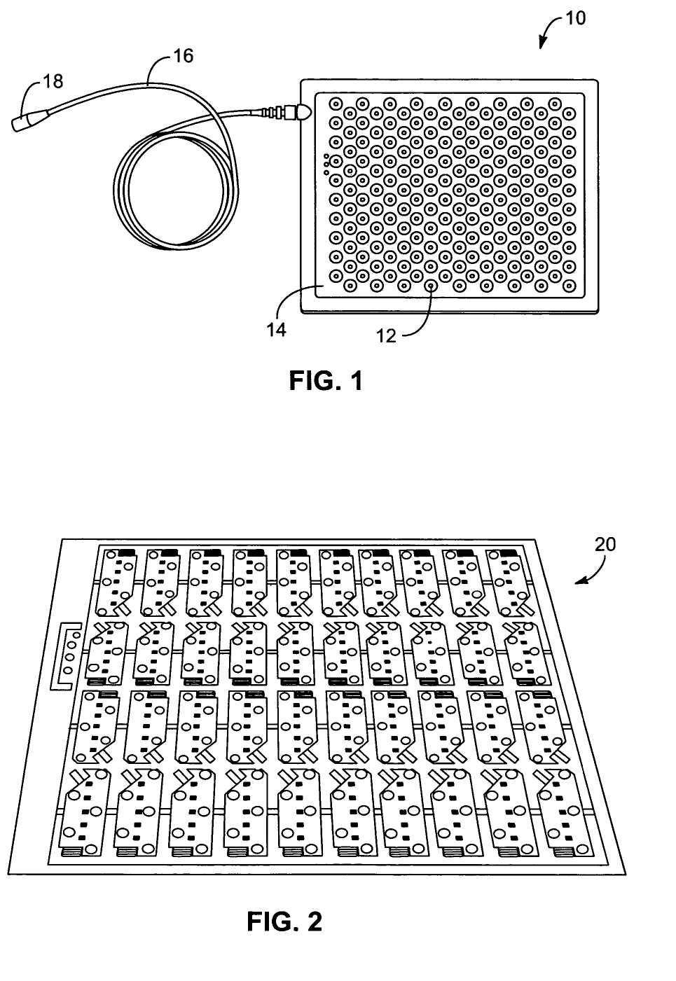

FIG. 1 and the corresponding discussion are intended to provide a general description of a representative embodiment of the present invention. One skilled in the art will appreciate that the invention may be practiced in a variety of system configurations to administer light therapy to a patient.

With reference to FIG. 1, a dynamic, flexible light pad is illustrated that is configured for administration of light therapy in accordance with a representative embodiment of the present invention. In FIG. 1, light pad 10 includes a plurality of light sources 12 that are spaced apart to administer or otherwise provide light therapy to a surface area of a patient.

Those skilled in the art will appreciate that the representative embodiment of FIG. 1 illustrates a representative light pad size in accordance with an embodiment of the present invention. Other embodiments of the present invention embrace other light therapy pads having a smaller or larger surface area for administering light therapy. Additionally, embodiments of the present invention embrace other configurations of light pads that administer light therapy in accordance with the present invention.

Light pad 10 further includes housing 14, cord 16, and connector 18. Housing 14 is configured to contain the plurality of light sources 12, which are connected to a flexible, electrical circuit board. In at least some embodiments, the light sources are light emitting diodes (LEDs).

In the illustrated embodiment of FIG. 1, housing 14 comprises an insulating material. Examples of insulating materials include a polymer material, urethane, foam, rubber, elastomers, ejection molding elastomer, and other insulating materials. In the present embodiment, housing 14 further includes a conductive component or layer independent of the flexible, electrical circuit board configuration, which collects and distributes heat and components of the flexible circuit board. In some embodiments, the conductive component is integrated into the flexible housing and/or integrated into the flexible, electrical circuit board configuration to collect and distribute heat or otherwise provide thermal conductivity. For example in one embodiment, the conductive component is integral to the housing, it being impregnated with heat conducting aluminum particles. In some embodiments, the conductive component or layer provides thermal contact with one or more light sources.

Connector 18 is configured to selectively couple to a power source (not shown). Cord 16 electrically couples the power source to the flexible, electrical circuit board of light pad 10 in order to eliminate the plurality of light sources 12 in providing light therapy. In one embodiment, indicator lights are provided to provide power indications such as when the light sources emit light that is not in the visible spectrum.

In some embodiments, the light therapy pad provides flexibility in at least two directions. In some embodiments, the light therapy pad is vacuum sealed, includes an air tight enclosure, and/or is water proof. In some embodiments, the dosage of light therapy is driven by the application time and/or the intensity of the light sources. In some embodiments, the application time is under one minute. In other embodiments, the application exceeds one minute. In some embodiments, the light therapy pad includes a feedback control mechanism that measures current output. For example, an open or closed loop controller controls the current driving the light therapy pad.

In some embodiments, temperature sensing circuitry and/or a resistor network for pad identification is provided. Such are connected, for example, back through a power cord and sensed by a controller.

Those skilled in the art will appreciate that embodiments of the present invention embrace a variety of wavelengths for the light therapy. For example, in some embodiments a light therapy pad of the present invention drives light at different wave lengths depending on the type of treatment to be applied to the patient. Examples of light therapy treatment in accordance with at least some embodiments of the present invention include use of the systems and/or methods of the present invention for the, treatment of an injury, tension headaches, chronic pain, migraine headaches, tension headaches, atypical facial pain, TMJ disorders, occipital neuralgia, neck-shoulder pain, fibromyalgia, medial epicondylitis, lateral epicondylitis, carpal tunnel syndrome, osteoarthritis, rheumatoid arthritis, pain and/or stiffness associated with arthritis, muscle spasm, costochondritis, spondylitis, low back strain, joint pain, sciatica, achilles tendonitis, ankle sprain, plantar fasciitis, shingles, Raynaud's Syndrome, reflex sympathetic dystrophy (also known as chronic regional pain syndrome), postherpetic neuralgia, burns, inflammation, pain, muscle spasm, wound healing, and the like. In some embodiments, a light therapy pad comprises one or more light sources that emit light of a first wavelength and one or more light sources that emit light at a second wavelength.

Light pad 10 of FIG. 1 provides a light therapy power density of approximately 8 mW/cm². However, those skilled in the art will appreciate that embodiments of the present invention embrace a light therapy power density that is less than or more than 8 mW/cm². Those skilled in the art will also appreciate that the size and shape of the light therapy pad illustrated in FIG. 1 is representative only. Embodiments of the present invention embrace other sizes and/or shapes of dynamic light therapy pads that are configured to apply light therapy to a patient.

With reference now to FIG. 2, a representative circuit board is illustrated that has been manufactured for use in light pad 10 of FIG. 1. As illustrated in FIG. 2, a manufactured printed circuit board 20 includes a plurality of smaller circuit boards that can be interconnected to provide increased flexibility, as will be further explained below. Printed circuit board 20 further includes various locations for components of an electrical circuit, including light sources for use in providing light therapy.

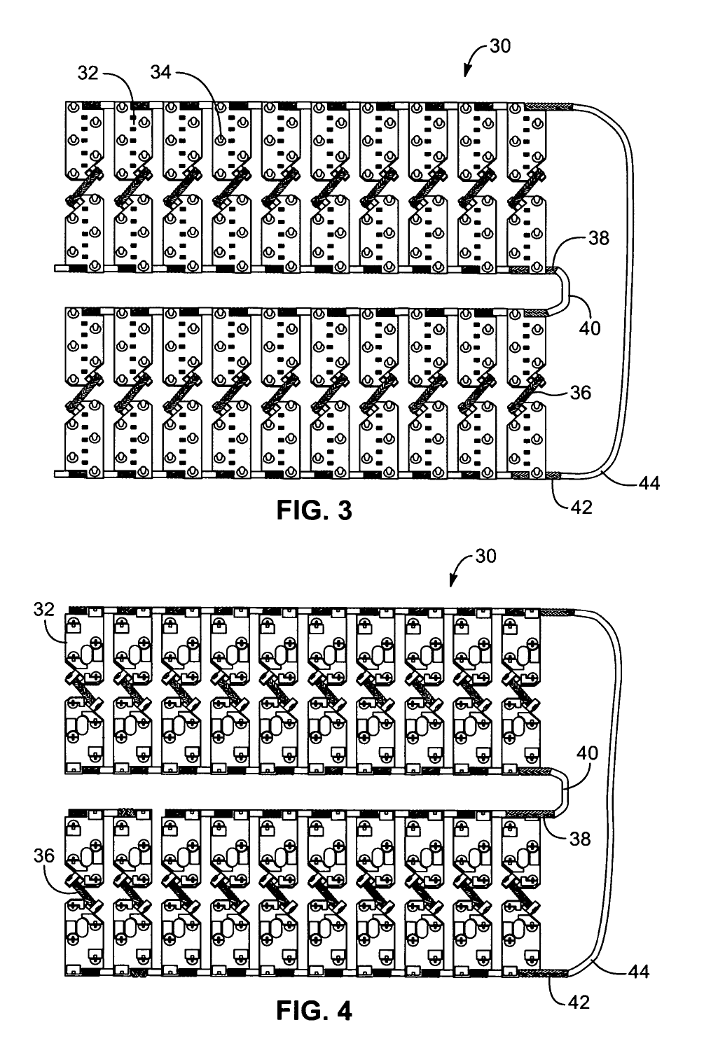

With reference now to FIG. 3, a top view of a flexible electrical circuit board configuration is illustrated, wherein the flexible circuit board configuration comprises a: plurality of circuit boards interconnected by a cord for utilization in a dynamic light pad in accordance with an embodiment of the present invention. In FIG. 3, flexible circuit board 30 includes a plurality of circuit boards 32, each having a plurality of light sources 34. In the illustrated embodiment, boards 32 are interconnected by use of a conductive cord 36. In some embodiments, conductive cord 36 is a stranded cord. In other embodiments, conductive cord 36 is a braided cord.

Boards 32 are further interconnected by conductive cord 38 and by a conductive cord 42. In the representative embodiment of FIG. 3, portions of conductive cord 38 are covered by an insulating covering 40. Similarly, portions of conductive cord 42 are covered by an insulating covering 44.

In the electrical circuit illustrated in FIG. 3, conductive cord 42 serves as a connection to the power source, while conductive cord 38 serves as a ground. Flexible circuit board 30 is a representative example of an electrical circuit board configuration.

With reference now to FIG. 4, in bottom view of a flexible, electrical circuit board configuration of FIG. 3 is illustrated, wherein the board comprises a plurality of circuit boards interconnected by one or more cords for utilization in a dynamic light pad to provide light therapy in accordance with an embodiment of the present invention.

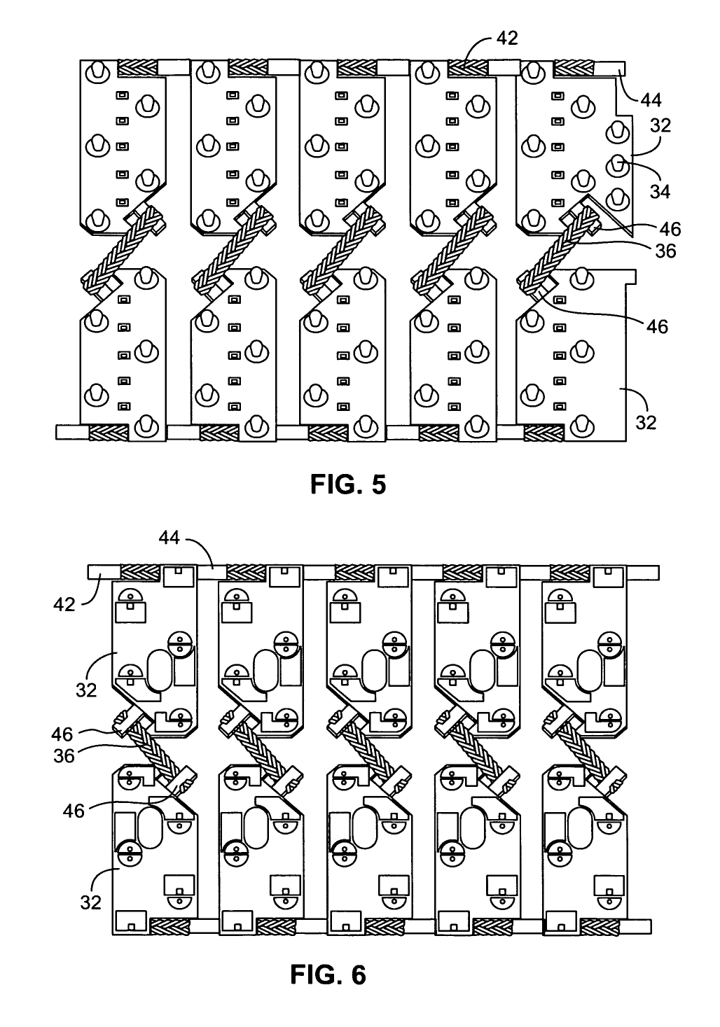

FIG. 5 illustrates a more detailed top view of the flexible, electrical circuit board configuration of FIG. 3. In FIG. 5, the illustrated embodiment includes a plurality of independent circuit boards 32, each having one or more light sources 34 and board connectors 46. It is further illustrated in FIG. 5 that conductive cord 36 interconnects multiple boards at corresponding board connectors 46. It is further illustrated that a technique for connecting multiple boards may include direct connection, such as is illustrated by conductive cord 36, or a weaving technique, such as is illustrated by conductive cord 42.

FIG. 6 illustrates a more detailed bottom view of the flexible electrical circuit board configuration of FIG. 3. Those skilled in the art will appreciate that a variety of techniques may be used to interconnect multiple boards to provide flexibility and durability in accordance with embodiments of the present invention. For example, insulated cords may be sewn, weaved, threaded, strung, braided, soldered or otherwise connected to interconnect independent circuit boards. The independent circuit boards provide increased flexibility for the light pad in a variety of directions to allow enhanced mapping to the contour of a patient's body. The stranded or weaved connecting conductors provide increased durability as the flexible circuit board is moved or shaped to a particular contour or configuration.

With reference back to FIG. 3, while the representative embodiment includes forty independent circuit boards, those skilled in the art will appreciate that embodiments of the present invention embrace more than forty circuit boards or less than forty circuit boards. Moreover, embodiments of the present invention embrace utilization of one or more rigid circuit boards and/or a flexible pc boards. In at least one embodiment of the present invention, the flexible circuit board is a single, flexible pc board. In another embodiment of the present invention, a plurality of independent flexible pc boards are utilized to provide a flexible circuit board for use in a light pad that is configured to provide light therapy to a patient.

In some embodiments, the flexible circuit board configuration comprises a flexible pc board that includes a flex circuit and/or a flex circuit with a lattice configuration.

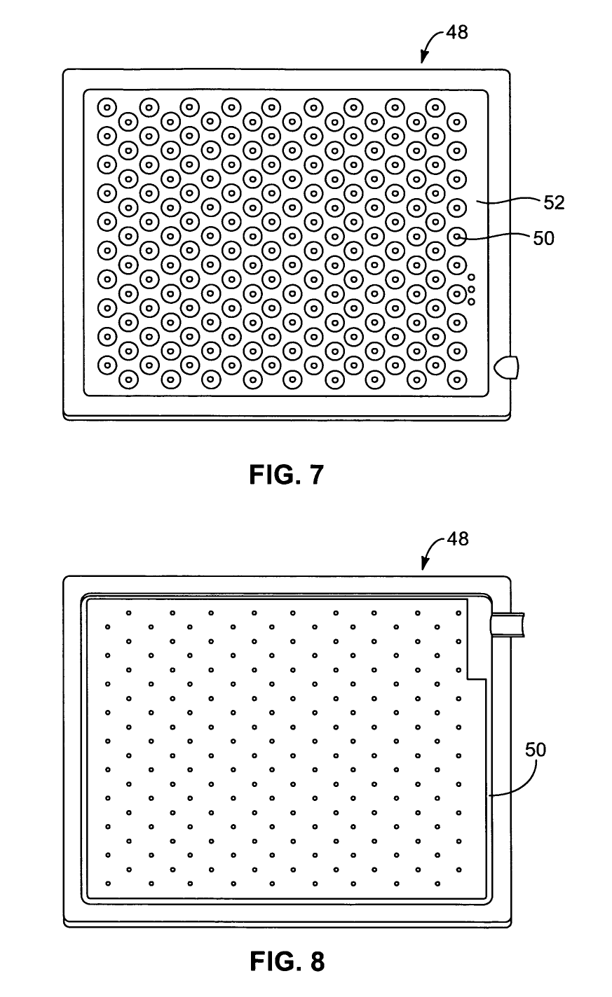

With reference now to FIG. 7, a top view of a dynamic light pad is illustrated in accordance with an embodiment of the present invention. In FIG. 7, top portion 48 of housing 14 (FIG. 1) comprises an insulating material that is formed to comprise a location 50 for each light source. Top portion 48 further includes a protective covering layer 52, which covers the plurality of light sources and provides a protective material between the patient and each light source.

In FIG. 8, a bottom view of representative top portion 48 of housing 14 (FIG. 1) is illustrated. In FIG. 8 an aperture is provided for each light source to enable each light source to be positioned in each corresponding location 50.

Thus, as discussed herein, the embodiments of the present invention embrace a light therapy device. In particular, the present invention relates to systems and methods for providing a light pad configured for light therapy, wherein the light pad is dynamically flexible to conform to curvatures of the human body.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A light therapy pad comprising:
a flexible, electrical circuit board configuration;
a plurality of light sources coupled to said flexible, electrical circuit board configuration, wherein said plurality of light sources are configured to provide light therapy to a patient; and
an electrical circuit comprising said plurality of light sources and electrical interconnections of said flexible, electrical circuit board configuration.

2. A light therapy pad as recited in claim 1, wherein said flexible electrical circuit board configuration comprises a plurality of circuit boards interconnected by a conductive cord.

3. A light therapy pad as recited in claim 2, wherein said conductive cord is a braided cord.

4. A light therapy pad as recited in claim 2, wherein said conductive cord is a stranded cord.

5. A light therapy pad as recited in claim 1, wherein said flexible, electrical circuit board configuration comprises at least one flexible pc board.

6. A light therapy pad as recited in claim 1, wherein said flexible, electrical circuit board configuration comprises a plurality of rigid circuit boards.

7. A light therapy pad as recited in claim 1, further comprising a housing configured to contain said flexible, electrical circuit board configuration.

8. A light therapy pad as recited in claim 7, wherein said housing comprises an insulating material.

9. A light therapy pad as recited in claim 8, wherein said insulating material comprises a polymer.

10. A light therapy pad as recited in claim 8, wherein said housing further, comprises a thermal conductive component that is configured to receive and distribute heat from one or more components of said electrical circuit.

11. A light therapy pad as recited in claim 7, wherein said housing includes a protective covering that prevents said plurality of light sources from coming in contact, with said patient.

12. A light therapy pad as recited in claim 1, wherein the light therapy is for at least one of:
(i) chronic pain;
(ii) a tension headache;
(iii) a migraine headache;
(iv) atypical facial pain;
(v) a TMJ disorder;
(vi) fibromyalgia;
(vii) osteoarthritis;
(viii) rheumatoid arthritis;
(ix) pain associated with arthritis;
(x) stiffness associated with arthritis;
(xi) increasing local blood circulation;
(xii) muscle spasm;
(xiii) joint pain;
(xiv) inflammation;
(xv) Raynaud's Syndrome;
(xvi) reflex sympathetic dystrophy;
(xvii) a burn;
(xviii) occipital neuralgia;
(xix) neck-shoulder pain;
(xx) frozen shoulder;
(xxi) medial epicondylitis;
(xxii) lateral epicondylitis;
(xxiii) carpal tunnel syndrome;
(xxiv) costochondritis;
(xxv) spondylitis;
(xxvi) low back strain;
(xxvii) sciatica;
(xxviii) hip arthritis;
(xxix) knee arthritis;
(xxx) an injury;
(xxxi) a post surgical procedure;
(xxxii) a post traumatic procedure;
(xxxiii) achilles tendonitis;
(xxxiv) an ankle sprain;
(xxxv) plantar fasciitis;
(xxxvi) shingles; and
(xxxvii) postherpetic neuralgia.

13. A light therapy device comprising:
a flexible circuit board for use in a light pad that is configured to provide light therapy to a patient;
a plurality of light sources coupled to said flexible circuit board;
an electrical circuit comprising said plurality of light sources and electrical interconnections of said flexible circuit board; and
a housing configured to contain said flexible circuit board, wherein said housing further comprises a thermal conductive component that is configured to receive and distribute heat from one or more components of said electrical circuit.

14. A light therapy device as recited in claim 13, wherein said flexible electrical circuit board configuration comprises a plurality of circuit boards interconnected by a conductive cord.

15. A light therapy device as recited in claim 14, wherein said conductive cord is a braided cord.

16. A light therapy device as recited in claim 14, wherein said conductive cord is a stranded cord.

17. A light therapy device as recited in claim 13, wherein said flexible, electrical circuit board configuration comprises at least one flexible pc board.

18. A light therapy device as recited in claim 13, wherein said flexible, electrical circuit board configuration comprises a plurality of rigid circuit boards.

19. A light therapy device as recited in claim 13, wherein said housing comprises an insulating material.

20. A light therapy device as recited in claim 13, wherein said insulating material comprises a polymer.

21. A method for manufacturing a device for administering light therapy to a patient, the method comprising:
providing a flexible circuit board;
coupling an electrical circuit to said flexible circuit board, said electrical circuit comprising a plurality of electrically connected components, including a plurality of light sources;
containing said flexible circuit board and said electrical circuit in a housing, said housing comprising an insulating material;
coupling said electrical circuit to an electrical connector that is configured to be coupled to a power source; and
providing said device for use in administering light therapy.

22. A method as recited in claim 21, further comprising:
providing a thermal conductive component that is configured to receive and disburse heat from said components; and
containing said thermal conductive component in said housing, wherein, said housing comprises an insulating material.

23. A method as recited in claim 21, wherein said providing a flexible circuit board includes:
providing a plurality of circuit boards; and
using a conductive cord to electrically interconnect said circuit boards.

24. A method as recited in claim 23, wherein said conductive cord is a braided cord.

25. A method as recited in claim 23, wherein said conductive cord is a stranded cord.

26. A method as recited in claim 21, wherein said providing a flexible circuit board comprises providing at least one flexible pc board.

27. A method as recited in claim 21, wherein the light therapy is for at least one of:
(i) chronic pain;
(ii) a tension headache;
(iii) a migraine headache;
(iv) atypical facial pain;
(v) a TMJ disorder;
(vi) fibromyalgia;
(vii) osteoarthritis;
(viii) rheumatoid arthritis;
(ix) pain associated with arthritis;
(x) stiffness associated with arthritis;
(xi) increasing local blood circulation;
(xii) muscle spasm;
(xiii) joint pain;
(xiv) inflammation;
(xv) Raynaud's Syndrome;
(xvi) reflex sympathetic dystrophy;
(xvii) a burn;
(xviii) occipital neuralgia;
(xix) neck-shoulder pain;
(xx) frozen shoulder;
(xxi) medial epicondylitis;
(xxii) lateral epicondylitis;
(xxiii) carpal tunnel syndrome;
(xxiv) costochondritis;
(xxv) spondylitis;
(xxvi) low back strain;
(xxvii) sciatica;
(xxviii) hip arthritis;
(xxix) knee arthritis;
(xxx) an injury;
(xxxi) a post surgical procedure;
(xxxii) a post traumatic procedure;
(xxxiii) achilles tendonitis;
(xxxiv) an ankle sprain;
(xxxv) plantar fasciitis;
(xxxvi) shingles; and
(xxxvii) postherpetic neuralgia.
